(19) United States
(12) Patent Application Publication
(10) Pub. No.: US 2019/0117869 A1
(43) Pub. Date: Apr. 25, 2019

(54) APPARATUS AND METHODS TO REMOVE TOXINS FROM BLOOD BY PLASMAPHERESIS

(71) Applicant: DD Innovations Lab, LLC, Neptune, NJ (US)

(72) Inventors: Garry Handelman, Neptune, NJ (US); Nathan Levin, Neptune, NJ (US); Sivanesan Dakshanamurthy, Neptune, NJ (US)

(73) Assignee: DD Innovations Lab, LLC, Neptune, NJ (US)

(21) Appl. No.: 16/169,199

(22) Filed: Oct. 24, 2018

Related U.S. Application Data
(60) Provisional application No. 62/576,394, filed on Oct. 24, 2017.

(51) Int. Cl. A61M 1/34; A61M 1/36

(52) U.S. Cl. CPC A61M 1/3486; A61L 2/022; A61M 1/3621

(57) ABSTRACT

Devices and method are provided for removing toxins, including protein-bound toxins, from blood. The device includes a housing having a receiving space and at least one hollow fiber extending through at least a portion of the receiving space. The at least one hollow fiber is operable to receive the stream of blood. The device further includes a plurality of beads disposed within the receiving space and external to the at least one hollow fiber. The at least one hollow fiber includes a plurality of pores operable to retain the one or more cellular elements of the stream of blood within the hollow fiber and to allow the one or more plasma elements of the stream of blood to pass from the hollow fiber to the receiving space of the housing. Each of the plurality of beads is configured to receive a toxin molecule from the one or more plasma elements.

### CROSS-REFERENCE TO RELATED APPLICATION

This application claims priority from U.S. Provisional Application No. 62/576,394, entitled “Method for Removal of Protein-Bound Toxins from Blood by Resin-Affinity Plasmapheresis,” filed on Oct. 24, 2017, which is incorporated by reference in its entirety, for all purposes, herein.

### BACKGROUND

### 1. Field of Technology

The present disclosure is generally directed to methods and apparatus for removing toxins from blood. The present disclosure is further directed to apparatus and methods for removing toxins, including protein-bound toxins, from blood using resin-affinity plasmapheresis.

### 2. Discussion of Related Technology

Conventional hemodialysis technology does not efficiently remove certain toxic molecules from blood, including, for example, toxins that are bound to proteins, such as albumin. Typical cartridge design is restricted to efficient clearance only of those molecules that are free in solution, such as unbound toxins. Dialysis cartridges generally utilize primarily diffusive clearance for those unbound molecules which occurs when the bloodstream passes through the dialyzer. The dialyzer is very efficient at removing these unbound molecules, such as urea and creatinine. For instance, 60-80% of urea and creatinine molecules can be removed by a single passage of blood through the dialyzer. Conventional hemodialysis has excellent efficiency for removal of molecules in the MW range from 50 Daltons to 5000 Daltons, under the condition that molecules which are free in solution are can diffuse through pores in the fibers. However, if the toxin molecules are protein-bound, certain challenges for toxin removal are created, as described further below.

The structure of the kidney is efficient for the removal of unbound toxins, through the glomerular filters. During normal function of the kidney, these unbound toxins are carried into the glomerular filtrate by convective transport across the glomerular basement membrane, and then are removed from the body with the ultrafiltrate in the formation of urine. However, the situation is different for toxins that are bound to proteins such as albumin. Protein-bound toxins such as albumin-bound toxins are removed by the kidney within the post-glomerular capillaries. When the blood passes through the kidney capillary network, the albumin molecules directly contact transport proteins on the wall of the capillary, which bind and remove the toxins, and add the toxin to the kidney ultrafiltrate. The standard dialyzer used in hemodialysis does not have this feature.

Plasmapheresis is a method using a modified dialyzer cartridge with larger pore sizes in the fibers, which allows proteins to pass through dialyzer fibers, into the external space around the fibers. These fibers keep red and white cells and other formed elements of blood inside the hollow center of the fiber, so that only proteins and other low-MW components reach the external compartment that surrounds the fibers. The plasmapheresis cartridge is therefore specifically engineered for the purposes of obtaining a concentrated protein ultrafiltrate. When the side ports on the plasmapheresis cartridge are closed, the device functions such that when blood is passed through the device under moderate pressure, the plasma protein ultrafiltrate leaves through the pores in the fibers, enters the external chamber, and then returns through the pores to the blood circuit.

Using conventional dialysis, the single pass clearance of these albumin-bound toxins during passage through the dialyzer is typically about 5-10%. As a result of this inefficient clearance, these toxic molecules accumulate to much higher levels in the tissues of patients with end stage renal disease. Levels in these patients can be 20 to 100 times higher than in individuals with normal renal function. Improved dialysis technology is needed to remove the albumin-bound toxins.

The need for these clearance mechanisms in the kidney capillaries is because these toxic substances carried on albumin cause injury to cells such as cardiac myocytes and other sensitive tissues. There are other classes of unbound molecules that are not efficiently removed by conventional hemodialysis. For example, in premature infants, there is often the accumulation of bilirubin, which may result in a condition called kernicterus. The bilirubin is carried on the plasma albumin of the neonate. This bilirubin has been formed by the accelerated hemolysis that occurs with prematurity. Neonatal intensive care units use specialized high-intensity blue light apparatus to accelerate the conversion of bilirubin to a nontoxic metabolite. However, improved methods, including hemodialysis methods for the removal of bilirubin from the blood of a patient is desirable.

Additionally, the removal of toxic drugs or ingested toxic substances that are bound to proteins, including albumin, is a major clinical challenge. Improved methods, including hemodialysis methods, for the removal to toxic drugs and ingested toxic substances from the blood of a patient is desirable. In particular, methods and apparatus for removing protein-bound toxic drugs and protein-bound ingested toxic substances from the blood of a patient is desirable. Such hemodialysis apparatus and techniques could be readily implemented in the intensive care unit to remove toxic drugs and toxic ingested substances from the blood of patients.

### BRIEF DESCRIPTION OF THE DRAWINGS

Embodiments of the present application are described, by way of example only, with reference to the attached Figures, wherein:

FIG. 1 is an illustration depicting a device for removing one or more toxin molecules from a stream of blood that includes a single hollow fiber, according to an example embodiment of the present disclosure;

FIG. 2 is an illustration depicting a cross-sectional view of a device for removing one or more toxin molecules from a stream of blood that includes a hollow fiber array, according to an example embodiment of the present disclosure; and

FIG. 3 is an illustration depicting a bead configured to receive a toxin molecule from a stream of blood, according to an example embodiment of the present disclosure.

It should be understood that the various aspects are not limited to the depictions provided in the drawings.

### DETAILED DESCRIPTION

Various embodiments of the disclosure are discussed in detail below. While specific implementations are discussed, it should be understood that this is done for illustration purposes only. A person skilled in the relevant art will recognize that other procedures and techniques can be used without parting from the spirit and scope of the present disclosure.

It should be understood at the outset that although illustrative implementations of one or more embodiments are illustrated below, the disclosed methods can be implemented using any number of techniques. The disclosure should in no way be limited to the illustrative implementations, drawings, and techniques disclosed herein, but can be modified within the scope of the appended claims along with their full scope of equivalents. In addition, numerous specific details are set forth in order to provide a thorough understanding of the embodiments described herein. However, it will be understood by those of ordinary skill in the art that the embodiments described herein can be practiced without these specific details. In other instances, methods, procedures and techniques have not been described in detail so as not to obscure the related relevant feature being described.

The following definitions are provided in order to aid those skilled in the art in understanding the present disclosure. As used herein, the term “bead” refers to any suitable particle having sufficient substrate surface area to effectively cause the transfer of toxins bound to plasma elements, such as plasma proteins, to the bead so as to carry out the presently disclosed techniques and methods.

The present disclosure provides devices and methods for removing toxins, including plasma protein bound toxins, from the blood stream. Typically plasma protein bound toxins are not efficiently removed by hemodialysis because such toxins are not carried free in the plasma. The present disclosure provides a solution to that problem.

According to at least one aspect of the present disclosure, a device for removing one or more toxin molecules from a stream of blood is provided. The device may include a housing having a receiving space and at least one hollow fiber extending longitudinally through at least a portion of the receiving space of the housing. The at least one hollow fiber may be operable to receive the stream of blood, which may include one or more cellular elements and one or more plasma elements. The device may also include a plurality of beads disposed within the receiving space and external to the at least one hollow fiber. The hollow fiber may include a plurality of pores operable to retain the one or more cellular elements of the stream of blood within the hollow fiber and to allow the one or more plasma elements of the stream of blood to pass from the hollow fiber to the receiving space of the housing. Each of the plurality of beads may be configured to receive a toxin molecule from the one or more plasma elements.

According to another aspect of the present disclosure, a method for removing one or more plasma protein-bound toxins from a blood stream is provided. The method may include passing a blood stream through at least one hollow fiber. The blood stream may include cellular elements and plasma proteins bound to a toxin molecule. The at least one hollow fiber may have a plurality of pores operable to retain the cellular elements within the hollow fiber and to allow plasma proteins bound to a toxin molecule to pass from the hollow fiber to a receiving space external to the hollow fiber. The method may further include causing the plasma proteins bound to a toxin molecule to contact a plurality of beads in the receiving space. The plurality of beads may be configured to chemically receive the toxin molecule from the plasma proteins, such that at least a portion of the toxin molecules bound to the plasma proteins is transferred to the plurality of beads thereby generating toxin-free plasma protein. The method may further include causing the toxin-free plasma protein to pass back to the hollow fiber to form a purified blood stream.

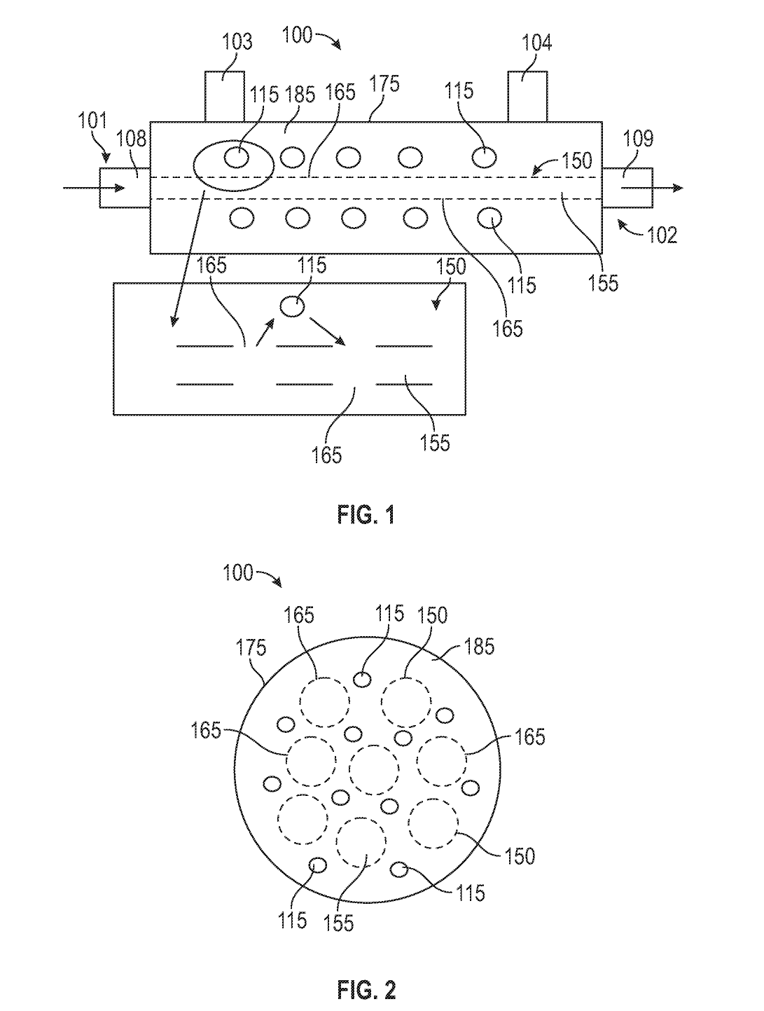

FIG. 1 is a longitudinal illustration of a device 100 for removing one or more toxin molecules from a stream of blood, according to an example embodiment of the present disclosure. As depicted in FIG. 1, device 100 includes a housing 175 having a receiving space 185. Device 100 further includes a hollow fiber 150 extending longitudinally through the receiving space 185 of housing 175. As depicted in FIG. 1, device 100 also includes a plurality of beads 115 disposed within the receiving space 185 and external to the hollow fiber 150.

The hollow fiber 150 is operable to receive a stream of blood from an inlet 108 at a first end 101 of device 100 into the inner bore 155 of hollow fiber 150. The stream of blood typically includes both cellular elements and plasma elements. Cellular elements may include red blood cells (RBCs), white blood cells (WBCs), and platelets. The plasma elements of the blood stream may include water, ions, organic molecules, trace elements, vitamins, and gases. The organic molecules in the plasma element component of the blood stream may include amino acids, proteins, glucose, lipids, and nitrogenous wastes. Typical proteins that may make up the plasma elements of the blood stream include albumins, globulins, and fibrinogen.

As depicted in FIG. 1, the hollow fiber 150 may include a plurality of pores 165 that are operable to retain the cellular elements of the blood stream within the inner bore 155 of hollow fiber 150 while allowing the plasma elements of the blood stream to pass from the hollow fiber 150 to the receiving space 185 of the housing 175. Therefore, hollow fiber 150 permits the plasma elements of the blood stream to flow into the receiving space 185 and contact the plurality of beads 115. The plurality of beads 115 are configured to chemically receive a toxin molecule that is bound to a plasma element such as a plasma protein. For instance, each of the plurality of beads 115 may include one or more chemical moieties capable of chemically receiving a toxin molecule that is bound to a plasma element, such as a plasma protein. In at least some instances, the chemical moiety may be covalently attached to a surface of each of the plurality of beads 115. In some instances, the chemical moiety may be a protein selected to cause the transfer of a toxin bound to a plasma element, such as a plasma protein, from a plasma element to the bead 115. For instance, the bead 115 may be coupled with a chemical moiety or protein that has a chemical affinity for the toxin molecule that is greater than or equal to the chemical affinity that the plasma element or plasma protein exhibits towards toxin molecule. In such cases, when a plasma element or plasma protein having a bound toxin molecule contacts the plurality of beads 115, the toxin molecule will transfer from the plasma element or plasma protein to the plurality of beads 115. In at least some instances, the chemical moiety may be albumin.

During operation of device 100, a blood stream having one or more plasma protein-bound toxins may be passed through hollow fiber 150. The cellular elements of the blood stream are retained within the inner bore 155 of hollow fiber 150 while the plasma elements, including plasma protein-bound toxins, may temporarily pass through pores 165 to the receiving space 185 within the housing 175. In at least some instances, the plasma protein may be albumin bound to a toxin. Once the plasma protein-bound toxins are within the receiving space 185, they may contact the plurality of beads 115 such that at least a portion of the toxin molecules bound to the plasma proteins is transferred to the plurality of beads thereby generating toxin-free plasma protein. In particular, toxic molecules that may be bound to plasma proteins, such as albumin, are transferred to the surface of the beads 115. The toxin-free plasma protein may then pass back to the hollow fiber 150 through one or more of pores 165 to rejoin the blood stream in order to form a purified blood stream. Once the plasma proteins pass back to the hollow fiber 150, the toxins remain bound to the beads 115. The purified blood stream may then exit device 100 at outlet port 109 proximal to a second end 102 of device 100. The blood stream may be passed back through device 100 at inlet port 108 several times during each treatment of a patient's blood providing for efficient toxin removal by the resin. The toxins that may be removed from the blood stream by device 100 may include urea, creatinine, indoxyl sulfate, bilirubin, a pharmaceutical drug, an ingested toxic substance, and uremic toxins.

Device 100 may be used as part of a dialysis system or plasmapheresis system in the treatment of a patient in need thereof. During the treatment, the patient's complete blood volume is circulated several times through the inlet 108 of device 100, as is done with conventional hemodialysis. During transit along the hollow fiber 150, the protein elements of the blood can exit the fiber and then return to the inside of the fiber. While present in the space outside the fiber, the protein elements interact with the resin and transfer toxins to the beads 115. The proteins then transit back to the inside of the hollow fiber 150. After the blood inside the hollow fiber 150 returns back to the patient, the toxins remain attached to the resin, thus achieving the objective of the device.

The pores 165 of hollow fiber 150 may have any diameter and characteristics so long as pores 165 cause the cellular elements of the blood stream to be retained in the hollow fiber 150 while permitting the passage of the plasma elements to pass back and forth between the hollow fiber 150 and the receiving space 185. The pores 165 may have an average diameter of from about 100 nanometer to about 1 micron, or from about 650 nanometers to about 1 micron, or from about 350 nanometers to about 1 micron, or from about 400 nanometers to about 700 nanometers. The porous hollow fiber 150 prevents contact of the cellular elements with the beads 115 thereby enhancing the biocompatibility of device 100 and increasing the effectiveness of toxin removal and decreasing fouling.

Device 100 may be coupled to one or more pumps (not shown) configured to cause the blood stream to pass through hollow fiber 150 and device 100. As depicted in FIG. 1, device 100 may also include ports 103, 104 which provide access to the receiving space 185 around hollow fiber 150. In at least some instances, ports 103, 104 may be opened and closed so that they may be used to insert the plurality of beads 115 into the receiving space 185 of device 100. Ports 103, 104 may then be closed to seal off the fluid flow path before use.

According to at least one aspect of the present disclosure, a method of preparing a hemodialysis cartridge or a plasmapheresis cartridge is provided. The method may include providing a cartridge such as device 100 depicted in FIG. 1 followed by inserting a plurality of beads, such as beads 115 depicted in FIG. 1, within the receiving space 185 of the hemodialysis cartridge or the plasmapheresis cartridge via ports, such as ports 103, 104. In at least some instances, conventional hemodialysis cartridges and/or plasmapheresis cartridges may be prepared using this method to form a device capable of the presently disclosed methods and techniques.

FIG. 2 illustrates a cross-sectional view of device 100 that includes hollow fiber 150 in the form of a hollow fiber array. As depicted in FIG. 2, device 100 includes hollow fibers 150, each having an inner bore 155, disposed within receiving space 185 in housing 175. A plurality of beads 115 are disposed within the receiving space 185 and external to the hollow fibers 150. Each hollow fiber 150 in the hollow fiber array includes a plurality of pores 165 configured to allow plasma elements to pass from the inner bore 155 of hollow fibers 150 to the receiving space 185 so as to contact the plurality of beads 115.

The beads 115 depicted in FIGS. 1 and 2 may be, for example, high-affinity resins capable of binding toxins found in the plasma component of blood, including those toxins bound to the plasma proteins of the blood stream. In some instances, the beads may be about 100 microns in diameter. In other cases, the beads may have an average diameter of from about 50 microns to about 500 microns, or from about 75 microns to about 125 microns.

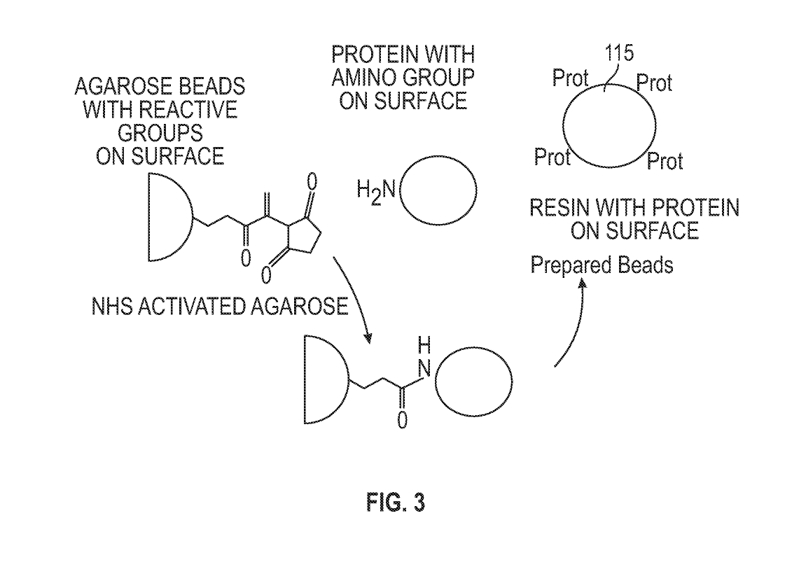

FIG. 3 illustrates a method of attaching chemical moieties capable of binding toxins to the beads, such as beads 115 depicted in FIGS. 1-2. As depicted in FIG. 3, bead 115 may be an agarose resin bead having reactive groups on the surface. In particular, FIG. 3 depicts the use of amino sepharose beads (1 micron in diameter) which contain a succinimide leaving group. The succinimide leaving group supports the equilibrium reaction that results in a stable linkage between the resin and the protein amino group. The beads are reacted with proteins that have reactive amino groups. This leads to covalent attachment of active proteins to the resin. The beads are then washed to remove chemicals used in the production process. The binding resin beads 115 shown in FIG. 3 will trap uremic toxins upon contact with plasma proteins having bound uremic toxins by means of collisional transfer between the plasma proteins and the resin binding sites.

Device 100 and the presently disclosed methods may be used to remove toxins bound to plasma proteins that are not efficiently removed by standard hemodialysis. In at least some instances, the presently disclosed methods and devices may be used to remove bilirubin bound to albumin in the blood of patients, such as in premature infants, in individuals with hepatic disease, and in individuals with greatly accelerated hemolysis of red blood cells. In such cases, a small volume hemodialysis device could accomplish this removal very quickly, if the albumin fraction of the plasma could be passed over beads, such as beads 115 shown in FIGS. 1-3, to which bilirubin would preferentially bind. Device 100 could remove much of the bilirubin within several hours, would be an alternative to exchange transfusion, and could in addition remove bilibubin carried on both plasma albumin and extracellular fluid albumin.

Additionally, the presently disclosed methods and techniques may be used in the removal of toxic drugs that are bound to albumin, which is a major clinical challenge using conventional hemodialysis methods and devices. The use of closed-circuit plasmapheresis, where the plasma fraction of the blood is passed over beads, such as those shown in FIGS. 1-3, that selectively binds toxic drugs, could be readily implemented in the intensive care unit. A similar approach could be applied to the removal of toxic substances that were accidentally ingested, and which bind to albumin. Accordingly, the present disclosed methods and devices may be used in the removal of protein-bound pharmaceutical drugs that reach toxic levels in the blood stream and to remove toxins that were absorbed from the diet in a patient not currently able to be removed by hemodialysis techniques.

### EXAMPLES

### Example 1

### Preparation of a Resin Bead Plasmapheresis Cartridge from a Commercial Cartridge

To prepare a plasmapheresis cartridge, a commercial cartridge was obtained which has ports that provide access to the space around the hollow fibers. After closure of one of the ports, a slurry of resin particles was pumped into the space around the hollow fibers. Following addition of this slurry to the space around the fibers, the other port was closed. The resultant device is a plasmapheresis cartridge with binding resin beads directly adjacent to the hollow fibers, in the space around the fibers. The cartridge is made of a large set of about 10,000 individual hollow fibers. Plasma proteins can exit the blood, and return to the blood, through the pores in the fiber, which are 1 micron in diameter. These pores enable the exit of proteins, which are 2-10 nm in diameter, but prevent the exit of cellular elements in the blood, which are more than 1 micron in diameter.

### Example 2

### Preparation of Resin Beads Having Active Albumin Surfaces

Agarose beads (50-150 microns in diameter) were treated with cyanogen bromide at pH 11 to form an activated resin with cyanate esters on the surface. The beads were washed with 0.1 M sodium bicarbonate, and bovine serum albumin was added in excess, followed by overnight incubation with stirring at 4° C. The free amino groups on the albumin reacted with the cyanate esters on the beads to form a covalent bond. The beads were centrifuged with an excess of bicarbonate to remove excess albumin. The final product typically had 10 mg of albumin per gram of packed beads. The beads were stored in distilled water.

In order to confirm activity, the beads were washed with 0.1M Na₂PO₄, pH 7.4, and 1 ml of beads were packed into a small syringe with a glass-wool plug. Then 1 ml of indoxyl sulfate, 100 μM in phosphate buffer, was slowly passed through the beads, and indoxyl sulfate measured by HPLC in the eluant. It was observed that 85% of the indoxyl sulfate was retained by the beads, due to binding to active sites on albumin.

### Example 3

### Removal of Indoxyl Solution From Aqueous Solution with a Plasmapheresis Column Containing Albumin Resin

Removal of indoxyl sulfate was tested in a plasmapheresis model system. Plasmapheresis columns were utilized with fiber surface area of 15 cm², and a pore size of 650 nanometers. The albumin resin, 1 ml, was introduced into the external compartment through a side port, and the device was closed and equilibrated with phosphate buffer. The resin is external to the fibers, and does not make direct contact with the interior of the fiber.

Indoxyl sulfate, 5 micromolar, 25 ml total volume, was passed through the device at 1 ml/minute, and the complete eluant was collected. With the side ports on the device closed, a portion of the indoxyl sulfate solution exited the fibers through the pores and contacted the resin. Because the system was closed, that solution returned to the fibers and exited the device.

The indoxyl sulfate concentration in the complete 25 ml eluant was reduced by 20%, as determined by HPLC. In a control experiment with an empty plasmapheresis cartridge, the indoxyl sulfate concentration was reduced 8%, consistent with dilution by the internal cartridge volume of 1.9 ml. On this basis, the net specific decrease of indoxyl sulfate by action of the albumin-coated resin was 12%.

### Statements of the Disclosure Include:

Statement 1: A device for removing one or more toxin molecules from a stream of blood, the device comprising: a housing having a receiving space; at least one hollow fiber extending longitudinally through at least a portion of the receiving space of the housing, the at least one hollow fiber operable to receive the stream of blood, wherein the stream of blood comprises one or more cellular elements and one or more plasma elements; and a plurality of beads disposed within the receiving space and external to the at least one hollow fiber; wherein the at least one hollow fiber comprises a plurality of pores, the plurality of pores operable to retain the one or more cellular elements of the stream of blood within the hollow fiber and to allow the one or more plasma elements of the stream of blood to pass from the hollow fiber to the receiving space of the housing; wherein each of the plurality of beads is configured to receive a toxin molecule from the one or more plasma elements.

Statement 2: A device according to Statement 1, wherein the one or more cellular elements comprises at least one selected from the group consisting of red blood cells, white blood cells, and platelets; and wherein the one or more plasma elements comprises at least one selected from the group consisting of free toxin molecules, amino acids, proteins, glucose, and lipids.

Statement 3: A device according to Statement 1 or Statement 2, wherein the one or more plasma elements comprises a protein.

Statement 4: A device according to any one of the preceding Statements 1-3, wherein the one or more plasma elements comprises a protein selected from the group consisting of albumins, globulins, and fibrogen.

Statement 5: A device according to any one of the preceding Statements 1-4, wherein the one or more plasma elements is albumin.

Statement 6: A device according to any one of the preceding Statements 1-5, wherein each of the plurality of beads comprises a chemical moiety capable of receiving a toxin molecule that is bound one or more plasma elements.

Statement 7: A device according to Statement 6, wherein the chemical moiety is covalently attached to a surface of each of the plurality of beads.

Statement 8: A device according to Statement 6 or Statement 7, wherein the chemical moiety is characterized by a greater than or equal chemical affinity toward the toxin molecule than the chemical affinity exhibited by the plasma element to the toxin molecule.

Statement 9: A device according to any one of the preceding Statements 1-8, wherein each of the plurality of beads comprises one or more proteins capable of receiving a toxin molecule from one or more plasma elements.

Statement 10: A device according to Statement 9, wherein the one or more proteins is covalently attached to a surface of each of the plurality of beads.

Statement 11: A device according to Statement 9 or Statement 10, wherein the one or more proteins is characterized by a greater chemical affinity toward the toxin molecule than the plasma element to which the toxin molecule is bound.

Statement 12: A device according to any one of the preceding Statements 1-11, wherein the one or more plasma elements comprises a toxin-bound plasma element bound to a toxin molecule, wherein each of the plurality of beads is configured to receive the toxin molecule from the toxin-bound plasma element.

Statement 13: A device according to any one of the preceding Statements 1-12, wherein each of the plurality of beads comprises one or more chemical moieties, each chemical moiety having a chemical affinity for the toxin molecule that is greater than or equal to the chemical affinity of the toxin-bound plasma element for the toxin molecule.

Statement 14: A device according to any one of the preceding Statements 1-13, wherein the one or more plasma elements comprises a plasma protein bound to a toxin molecule, wherein each of the plurality of beads is configured to receive the toxin molecule from the plasma protein.

Statement 15: A device according to any one of the preceding Statements 1-14, wherein each of the plurality of beads comprises one or more chemical moieties, each chemical moiety having a chemical affinity for the toxin molecule that is greater than or equal to the chemical affinity of the plasma protein for the toxin molecule.

Statement 16: A device according to any one of the preceding Statements 1-15, wherein the plasma protein is albumin.

Statement 17: A device according to any one of the preceding Statements 1-16, wherein the one or more chemical moieties is a protein covalently attached to a surface of each of the plurality of beads.

Statement 18: A device according to any one of the preceding Statements 1-17, wherein the one or more chemical moieties comprises albumin.

Statement 19: A device according to any one of the preceding Statements 1-18, wherein the toxin is selected from the group consisting of: urea, creatinine, indoxyl sulfate, bilirubin, a pharmaceutical drug, an ingested toxic substance, and uremic toxins.

Statement 20: A device according to any one of the preceding Statements 1-19, wherein the plurality of pores comprises an average diameter between about 100 nanometers and about 1 micron.

Statement 21: A device according to any one of the preceding Statements 1-20, wherein the plurality of pores comprises an average diameter between about 650 nanometers and about 1 micron.

Statement 22: A device according to any one of the preceding Statements 1-21, wherein the plurality of pores comprises an average diameter between about 350 nanometers and about 1 micron.

Statement 23: A device according to any one of the preceding Statements 1-22, wherein the plurality of pores comprises an average diameter between about 400 nanometers and about 700 nanometers.

Statement 24: A device according to any one of the preceding Statements 1-23, wherein the plurality of beads comprises an average diameter between about 50 microns and about 500 microns.

Statement 25: A device according to any one of the preceding Statements 1-24, wherein the plurality of beads comprises an average diameter between about 75 microns and about 125 microns.

Statement 26: A device according to any one of the preceding Statements 1-25, wherein the at least one fiber comprises a hollow fiber array.

Statement 27: A device according to any one of the preceding Statements 1-26, wherein the plurality of beads comprises agarose resin beads.

Statement 28: A device according to any one of the preceding Statements 1-27, wherein the plurality of beads comprises amino sepharose beads with a succinimide leaving group.

Statement 29: A method for removing one or more plasma protein-bound toxins from a blood stream, the method comprising: passing a blood stream through at least one hollow fiber, wherein the blood stream comprises cellular elements and plasma proteins bound to a toxin molecule, the at least one hollow fiber having a plurality of pores operable to retain the cellular elements within the hollow fiber and to allow plasma proteins bound to a toxin molecule to pass from the hollow fiber to a receiving space external to the hollow fiber; causing the plasma proteins bound to a toxin molecule to contact a plurality of beads in the receiving space, the plurality of beads configured to chemically receive the toxin molecule from the plasma proteins, such that at least a portion of the toxin molecules bound to the plasma proteins is transferred to the plurality of beads thereby generating toxin-free plasma protein.

Statement 30: A method for removing one or more toxins from the blood of a patient in need thereof, the method comprising: passing a blood stream through at least one hollow fiber, wherein the blood stream comprises cellular elements and plasma proteins bound to a toxin molecule, the at least one hollow fiber having a plurality of pores operable to retain the cellular elements within the hollow fiber and to allow plasma proteins bound to a toxin molecule to pass from the hollow fiber to a receiving space external to the hollow fiber; causing the plasma proteins bound to a toxin molecule to contact a plurality of beads in the receiving space, the plurality of beads configured to chemically receive the toxin molecule from the plasma proteins, such that at least a portion of the toxin molecules bound to the plasma proteins is transferred to the plurality of beads thereby generating toxin-free plasma protein.

Statement 31: A method according to Statement 29 or Statement 30, further comprising causing the toxin-free plasma protein to pass back to the hollow fiber, thereby forming a purified blood stream.

Statement 32: A method according to any one of the preceding Statements 29-31, wherein the plasma protein is albumin.

Statement 33: A method according to any one of the preceding Statements 29-32, wherein each of the plurality of beads comprises one or more chemical moieties capable of chemically receiving a toxin molecule that is bound to the plasma proteins.

Statement 34: A method according to Statement 33, wherein the one or more chemical moieties is covalently attached to a surface of each of the plurality of beads.

Statement 35: A method according to Statement 33 or Statement 34, wherein the one or more chemical moieties has a chemical affinity for the toxin molecule that is greater than or equal to the chemical affinity of the plasma protein for the toxin molecule.

Statement 36: A method according to any one of the preceding Statements 33-35, wherein the one or more chemical moieties comprises albumin.

Statement 37: A method according to any one of the preceding Statements 29-36, wherein the toxin molecule is selected from the group consisting of: urea, creatinine, indoxyl sulfate, bilirubin, a pharmaceutical drug, an ingested toxic substance, and uremic toxins.

Statement 38: A method according to any one of the preceding Statements 29-37, wherein the plurality of pores comprises an average diameter between about 100 nanometers and about 1 micron.

Statement 39: A method according to any one of the preceding Statements 29-38, wherein the plurality of pores comprises an average diameter between about 650 nanometers and about 1 micron.

Statement 40: A method according to any one of the preceding Statements 29-39, wherein the plurality of pores comprises an average diameter between about 350 nanometers and about 1 micron.

Statement 41: A method according to any one of the preceding Statements 29-40, wherein the plurality of pores comprises an average diameter between about 400 nanometers and about 700 nanometers.

Statement 42: A method according to any one of the preceding Statements 29-41, wherein the plurality of beads comprises an average diameter between about 50 microns and about 500 microns.

Statement 43: A method according to any one of the preceding Statements 29-42, wherein the plurality of beads comprises an average diameter between about 75 microns and about 125 microns.

Statement 44: A method according to any one of the preceding Statements 29-43, wherein the at least one hollow fiber comprises a hollow fiber array.

Statement 45: A method according to any one of the preceding Statements 29-44, wherein the plurality of beads comprises agarose resin beads.

Statement 46: A method according to any one of the preceding Statements 29-45, wherein the plurality of beads comprises amino sepharose beads with a succinimide leaving group.

Statement 47: A method of removing bilirubin from the blood of a patient in need thereof, the method comprising the method according to any one of Statements 29-46.

Statement 48: A method of treating or preventing kernicterus in a patient in need thereof, the method comprising the method according to any one of Statements 29-46.

What is claimed is:

1. A device for removing one or more toxin molecules from a stream of blood, the device comprising:
a housing having a receiving space;
at least one hollow fiber extending longitudinally through at least a portion of the receiving space of the housing, the at least one hollow fiber operable to receive the stream of blood, the stream of blood including one or more cellular elements and one or more plasma elements; and
a plurality of beads disposed within the receiving space and external to the at least one hollow fiber,
wherein,
the at least one hollow fiber includes a plurality of pores,
the plurality of pores are operable to retain the one or more cellular elements of the stream of blood within the hollow fiber and to allow the one or more plasma elements of the stream of blood to pass from the hollow fiber to the receiving space of the housing, and
each of the plurality of beads is configured to receive a toxin molecule from the one or more plasma elements.

2. The device according to claim 1,
wherein,
the one or more cellular elements comprises at least one selected from the group consisting of red blood cells, white blood cells, and platelets, and
the one or more plasma elements comprises at least one selected from the group consisting of free toxin molecules, amino acids, proteins, glucose, and lipids.

3. The device according to claim 2, wherein the one or more plasma elements comprises a protein.

4. The device according to claim 1, wherein the one or more plasma elements is albumin.

5. The device according to claim 1, wherein each of the plurality of beads comprises a chemical moiety capable of receiving a toxin molecule that is bound to one or more plasma elements.

6. The device according to claim 5, wherein the chemical moiety is characterized by a greater chemical affinity toward the toxin molecule than the chemical affinity exhibited by the plasma element to the toxin molecule to which it is bound.

7. The device according to claim 1,
wherein,
the one or more plasma elements comprises a plasma protein bound to a toxin molecule, and
each of the plurality of beads is configured to receive the toxin molecule from the plasma protein.

8. The device according to claim 7,
wherein,
each of the plurality of beads comprises one or more chemical moieties, and
each of the one or more chemical moieties have a chemical affinity for the toxin molecule that is greater than or equal to the chemical affinity of the plasma protein for the toxin molecule.

9. The device according to claim 8, wherein the plasma protein is albumin.

10. The device according to claim 9, wherein the one or more chemical moieties is a protein covalently attached to a surface of each of the plurality of beads.

11. The device according to claim 10, wherein the one or more chemical moieties comprises albumin.

12. The device according to claim 1, wherein the toxin is selected from the group consisting of:
urea, creatinine, indoxyl sulfate, bilirubin, a pharmaceutical drug, an ingested toxic substance, and uremic toxins.

13. The device according to claim 1, wherein the plurality of pores comprises an average diameter from about 100 nanometers to about 1 micron.

14. The device according to claim 1, wherein the at least one fiber comprises a hollow fiber array.

15. A method for removing one or more plasma protein-bound toxins from a blood stream, the method comprising:
passing a blood stream through at least one hollow fiber, wherein the blood stream comprises cellular elements and plasma proteins bound to a toxin molecule, the at least one hollow fiber having a plurality of pores operable to (i) retain the cellular elements within the hollow fiber, and (ii) allow plasma proteins bound to a toxin molecule to pass from the hollow fiber to a receiving space external to the hollow fiber;
causing the plasma proteins bound to a toxin molecule to contact a plurality of beads in the receiving space, the plurality of beads configured to chemically receive the toxin molecule from the plasma proteins such that at least a portion of the toxin molecules bound to the plasma proteins is transferred to the plurality of beads thereby generating toxin-free plasma protein.

16. The method according to claim 15, further comprising:
causing the toxin-free plasma protein to pass back to the hollow fiber to form a purified blood stream.

17. The method according to claim 15, wherein the plasma protein is albumin.

18. The method according to claim 15,
wherein,
each of the plurality of beads comprises one or more chemical moieties capable of chemically receiving a toxin molecule that is bound to the plasma proteins, and
the one or more chemical moieties have a chemical affinity for the toxin molecule that is greater than or equal to the chemical affinity of the plasma protein for the toxin molecule.

19. The method according to claim 18, wherein the one or more chemical moieties comprises albumin.

20. The method according to claim 15, wherein the toxin molecule is selected from the group consisting of: urea, creatinine, indoxyl sulfate, bilirubin, a pharmaceutical drug, an ingested toxic substance, and uremic toxins.
